(19) United States
(12) Patent Application Publication
(10) Pub. No.: US 2020/0215187 A1
(43) Pub. Date: Jul. 9, 2020

(54) VACCINE ADJUVANT AND MICRONEEDLE PREPARATION

(71) Applicant: BIOSERENTACH CO., LTD., Kyoto (JP)

(72) Inventor: Kanji TAKADA, Kyoto-shi, Kyoto (JP)

(73) Assignee: BIOSERENTACH CO., LTD., Kyoto (JP)

(21) Appl. No.: 16/632,983

(22) PCT Filed: Jul. 20, 2018
(86) PCT No.: PCT/JP2018/027247

§ 371 Date: Jan. 22, 2020

(30) Foreign Application Priority Data
Jul. 24, 2017 (JP) 2017-142472

(51) Int. Cl. A61K 39/39; A61K 47/36; A61M 37/00

(52) U.S. Cl. CPC A61K 39/39; A61K 9/0019; A61M 37/0015; A61K 47/36

(57) ABSTRACT

The problem to be solved of the present invention is to provide a new vaccine adjuvant. The means for solving the problem is a vaccine adjuvant including dextran sulfate or its derivatives.

### TECHNICAL FIELD

The present invention relates to a skin vaccine and a vaccine adjuvant.

### BACKGROUND ART

The major vaccine companies in the world are developing influenza vaccine using subcutaneous injection syringe attaching hollow metal microneedles. However, developments do not advance smoothly at all, because of high back pressure at the injection site and/or limited volume of the subcutaneously injected solution.

As an alternative skin vaccination to the subcutaneous injector, there is a microneedle preparation. Patent document 1 describes a three-layered dissolving microneedle preparation for the delivery of vaccine antigen to epidermis. The three-layered dissolving microneedle preparation disclosed in patent document 1 is a skin vaccine. This is formed by kneading a vaccine antigen with base. As the base, a polymer substance having thread-forming property and water-soluble property is used.

The thread-forming property means the property of polymer substance to become viscous and extended as a thread when it is dissolved with a small amount of water. The thread-forming property of the polymer substance can be ascertained by pulling the concentrated solution of polymer substance made by mixing with a small amount of water. The microneedle preparation of patent document 1 has the internal structure in which polymer entwines with each other. As a result, the microneedle preparation has enhanced strength, though being water-soluble, and can be used as an insertion preparation to the skin.

As the dissolving microneedle preparation is a solid preparation, it is superior to the subcutaneous injection preparation because of good long-term stability of vaccine antigen. With respect to performance of a skin vaccine using dissolving microneedle preparation, it is expected that immune induction ability is enhanced.

In general, the antibody titer can be induced more strongly by using a vaccine adjuvant in combination with a vaccine antigen. However, the conventionally known vaccine adjuvants are water-insoluble, low soluble or insufficient in thread-forming property.

As vaccine adjuvants, mineral acid salt such as aluminum hydroxide, and plant components such as toxin, emulsion and saponin etc. are known. However, when they are formulated in dissolving microneedles, they may affect the dissolving microneedle preparation in shape, strength and solubility.

Patent documents 2 describes a vaccine adjuvant containing polymeric polysaccharides and esters of fatty acid etc. However, patent documents 2 is silent with respect to adjuvant effect of an acid ester other than carboxylate esters.

When a substance having water-insoluble property, low soluble property or insufficient thread-forming property are contained in dissolving microneedles, strength or solubility of the dissolving microneedles may suffer adverse effect. For example, when dissolving microneedle preparation decreases in strength, there is a possibility that it may not withstand against paracentesis into the skin. Also, when dissolving microneedle preparation decreases in solubility, there is a possibility that it may not dissolve spontaneously with small amount of the body fluid existing in the skin.

### PRIOR ART DOCUMENTS

### Patent Document

Patent document 1: JP2012-90767 A
Patent document 2: JP2001-39891 A

### DISCLOSURE OF THE INVENTION

### Problem to be Solved by the Invention

The present invention solves the conventional problems, and an object thereof is to provide a new vaccine adjuvant.

### Means for Solving the Problem

Dextran sulfate is a polymer substance satisfying the two physicochemical properties that are thread-forming property and water-soluble property. In other words, when dextran sulfate is kneaded with a small amount of water to prepare hydrogel, it shows strong thread-forming property. In addition to thread-forming property and water-soluble property, it was found that dextran sulfate has high immunological activity.

The present invention provides a vaccine adjuvant including dextran sulfate or its derivatives.

Also, the present invention provides a dissolving microneedle preparation containing the vaccine adjuvant.

It is the above described dissolving microneedle preparation, further contains a vaccine antigen.

It is any one of the dissolving microneedle preparations having a plurality of layers laminated in the direction connecting between acral part and bottom part.

It is any one of the above described dissolving microneedle preparations, wherein the layer having acral part contains a vaccine antigen.

It is any one of the above described dissolving microneedles preparations, wherein the layer having bottom part does not contain a vaccine antigen.

It is any one of the dissolving microneedle preparations, wherein a layer, which is different from the layer containing vaccine antigen, contains the vaccine adjuvant.

It is any one of the dissolving microneedle preparations, wherein a layer, which is located lower in the direction to bottom than the layer containing vaccine antigen, contains the vaccine adjuvant.

It is any one of the dissolving microneedle preparations containing a polymer substance having thread-forming property and water-soluble property as base.

Also, the present invention provides a microneedles preparation having a film containing the vaccine adjuvant, formed on its surface.

Also, the present invention provides a percutaneously absorbable preparation having the vaccine adjuvant.

### Effect of the Invention

According to the present invention, a new vaccine adjuvant is provided. By using the vaccine adjuvant of the present invention, a dissolving microneedle preparation having high strength, high solubility and high immune induction ability is provided.

### BRIEF DESCRIPTION OF DRAWINGS

FIG. 1 It is an enlarged photograph of the dissolving microneedles prepared in Example 4.

FIG. 2 It is an enlarged photograph of the dissolving microneedles prepared in Comparative example 2.

### EMBODIMENT FOR CARRYING OUT THE INVENTION

Dextran sulfate means a sulfuric acid ester of dextran. The dextran sulfate may have, at a sulfate part, the structure of acid or the structure of salt. The dextran sulfate may be its derivatives. Examples of the dextran sulfate or its derivatives include sodium dextran sulfate, potassium dextran sulfate, dextran sulfate ester sodium sulfur 18 (official drug) etc.

As the dextran sulfate, one available from the market can be used. A commercial product thereof may be the product called “SODIUM DEXTRAN SULFATE” (trade name) sold by Nacalai Tesque Co., Ltd. as a chemical reagent. The other commercial products thereof may be the products sold by Meito Sangyo Co., Ltd. as a medical ingredient, a raw material of medical device or a chemical reagent, called “DEXTRAN SULFATE ESTER SODIUM SULFUR 18” (trade name), “DEXTRAN SULFATE ESTER SODIUM SULFUR 5” (trade name), DSH (trade name), DS 500 (trade name) and DST-H (trade name).

The molecular weight of dextran sulfate is not specifically limited. When dextran sulfate is used as base of a microneedle preparation, the molecular weight of dextran sulfate is properly determined in consideration of strength and solubility demanded for the microneedle preparation. In such case, for example, the molecular weight of dextran sulfate is about 1,000-100,000, preferably about 2,000-50,000, more preferably about 3,000-10,000 for weight average molecular weight (MW).

The molecular weight of the dextran sulfate which Meito Sangyo Co., Ltd. sells is about 4000-7000 which is a calculated value from the molecular weight of dextran and the sulfuric acid group substitution degree. The molecular weight of the sodium dextran sulfate which Nacalai Tesque Co., Ltd. sells as chemical reagent is about 5,000-6,000, about 500,000, or about 950,000-2,000,000.

Dextran sulfate can be used alone as a vaccine adjuvant. In such a case, the vaccine adjuvant is consisting of the dextran sulfate. Dextran sulfate may be used as a vaccine adjuvant in combination with the conventionally used additives, solvents etc.

For example, the additives used in combination with dextran sulfate include oil ingredients such as oleic acid, squalane, squalene, liquid paraffin, synthetic linear alkane based oil and alcohol fatty acid ester oil; surfactants such as polyglycerol ester; aluminum phosphate gel, aluminum hydroxide gel etc.

The vaccine adjuvant including dextran sulfate is formulated into various vaccine preparations according to their type of usage with the amount to effectively show their function. Type of the vaccine preparation, is not specifically limited, but include, for example, a subcutaneous injection preparation, percutaneously absorbable preparations such as a microneedle preparation, a patch preparation and a liniment.

A vaccine preparation of the present invention contains the vaccine adjuvant of the present invention and a desired antigen. The aqueous type vaccine preparations may be prepared by adding and mixing a desired antigen solution to the vaccine adjuvant of the present invention and, if necessary, emulsifying and/or suspending. Otherwise, they may be prepared by adding and mixing the vaccine adjuvant of the present invention into the aqueous type vaccine or oily type vaccine containing a desired antigen, if necessary, emulsifying and/or suspending them. As the desired antigen, any antigen may be used.

The content of dextran sulfate or its derivative in aqueous type vaccine preparations may vary depending on the dosage form, and is preferably 0.05-1.0 w/v %, and more preferably 0.1-0.5 w/v %.

Also, the content of desired antigen in vaccine is preferably 0.05-5.0 w/v %, and more preferably 0.1-1.0 w/v %.

### <Dissolving Microneedle Preparation>

A dissolving microneedle preparation of the present invention is manufactured, for example, by forming microneedles using a mold, and subsequently by fixing the formed microneedles to a supporting body. As a mold, board materials in which holes corresponding to configuration and conformation of the microneedles are made, is used. As materials of the board materials, fluoric resin, silicone resin or ABS resin etc. may be used.

At first, base which is a raw material of the microneedle, an objective substance and water etc. are mixed, and a raw material mixture is prepared. In the raw material mixture, the dextran sulfate and, if necessary, the additives etc. are included. It is desirable not to use oily additives, because the strength of microneedle becomes fragile when they are mixed with dextran sulfate.

In an embodiment of the present invention, dextran sulfate is used as base of the microneedles. The dextran sulfate and water-soluble and thread-forming polymer substance may be mixed and used as the base to adjust strength and solubility of the microneedles.

When the mixture of dextran sulfate and water-soluble thread-forming polymer substance is used as the base, dextran sulfate is used at an amount of preferably more than 50% by weight of the base. When the content of dextran sulfate becomes below 50% by weight of the base, immune induction ability of the dissolving microneedle preparation may decrease. The content of dextran sulfate in the base is preferably 60-95% by weight, and more preferably is 70-90% by weight.

Water-soluble and thread-forming polymer substance used as the base with dextran sulfate is at least one substance selected from the group consisting of polysaccharides, proteins, polyvinyl alcohol, carboxyvinyl polymers and sodium polyacrylate that have thread-forming property. In addition, with respect to these polymer substances, only one kind may be used, or several kinds may be used in combination.

Preferably, the thread-forming polysaccharides are at least one substance selected from sodium chondroitin sulphate, dextran, hyaluronic acid, cyclodextrin, hydroxypropyl cellulose, carboxymethylcellulose, alginic acid, agarose, pullulan and glycogen and derivatives thereof.

Preferably, the thread-forming protein is at least one substance selected from serum albumin, serum α-acid glycoprotein, collagen, low-molecular weight collagen and gelatin and derivatives thereof.

Particularly preferable water-soluble thread-forming polymer substances include sodium chondroitin sulfate, dextran, hyaluronic acid etc.

As objective substances of the dissolving microneedle preparation, vaccine antigen may be used. Examples of the vaccine antigen include those which are available from the market such as diphtheria vaccine antigen, tetanus vaccine antigen, whooping cough vaccine antigen, hepatitis vaccine antigen, Streptococcus pneumoniae vaccine antigen, influenza vaccine antigen and uterine cervix cancer vaccine antigen. In addition to these, HIV vaccine antigen, tuberculosis vaccine antigen, malarial vaccine antigen, high blood pressure vaccine antigen, diabetes vaccine antigen, Alzheimer's disease vaccine antigen, no-smoking vaccine antigen, obesity vaccine antigen, contraception vaccine antigen are cited, although not limited to these.

The content of objective substance, namely the content of antigen, in microneedle preparations is, though depending on the kind of antigen, lower than 50% by weight, preferably 0.1-40% by weight, and more preferably 1-30% by weight based on the solid of raw material composition making a single layer. When the content of antigen exceeds 50% by weight, strength of the microneedle preparation may decrease.

Then, the raw material mixture is put on the mold, and is filled into the holes made in the mold, if necessary, under embrocation pressure using an embrocation tool or an embrocation equipment. Centrifugal force may be applied to the mold using centrifuges etc. to make filling surely.

A supporting body is put on a mold so as to contact with the raw material mixture before the raw material mixture dries. The supporting body is porous, and it comes in contact with raw material mixture. Thereby, component of the raw material mixture penetrates into pores of the supporting body due to anchor effect. As a result, it bonds strongly, and, at the same time, is able to absorb and release water contained in the raw material mixture.

The supporting body consists of a material which is able to fix the microneedles strongly. The supporting body is water-insoluble. The supporting body has stiffness, and does not change its form under the room temperature environment.

A preferred supporting body is a molded body consisting of a water-insoluble excipient for tablets. This is because it has a good productivity, and is suitable for medical manufacturing processes such as sterilization. The excipient for tablets may be a composition containing several components. Examples of preferable excipient for tablets include cellulose acetate, crystalline cellulose, cellulose derivatives, chitin and chitin derivatives etc.

The molded body consisting of excipient for tablets may be manufactured according to the same manner as a tablet. For example, the excipient for tablets is put in a mortar of tableting machine, and molded by a pestle with a suitable tableting pressure. The size of supporting body is suitably adjusted by increasing or decreasing the diameter of the mortar, the amount to be filled of the excipient for tablets and tableting pressure.

Subsequently, the raw material mixture which have been filled into the holes is dried. The drying is performed at the temperature of less than 50° C., preferably lower than room temperature to prevent degradation of the objective substances. Thereafter, the supporting body is separated from the mold to obtain a microneedle array chip which has many dissolving microneedle preparations of the present invention implanted on the supporting body.

In an embodiment, several kinds of raw material mixtures are prepared from different component, and they are filled into the mold in turn when filled in the mold. According to the process, a dissolving microneedle preparation which has several layers composed of different component, laminated in the direction connecting between acral part and bottom part, is obtained. In such case, at least one layer of the laminated layers may include dextran sulfate as base. Also, it is preferable for at least one layer of the laminated layers contains vaccine antigen as objective substance.

It has been revealed that, when several kinds of raw material mixtures are filled into a mold, a raw material mixture including dextran sulfate is hard to mix with a raw material mixture not containing it, which is previously or later filled as base. As a result, a clear boundary surface between the two layers is formed. Namely, the dextran sulfate used as base is hard to be mixed with a component of the layers containing no dextran sulfate, which is filled previously or later, and will be mutually separated.

In a preferable embodiment of dissolving microneedle preparation having several layers, vaccine antigen is contained in the layer having acral part. A vaccine antigen is, thereby, easily delivered to the Dendritic cells located in the dermal layer. Vaccine adjuvant may be contained into the layer having acral part. Otherwise, it may be contained in a layer located lower in the direction to bottom part than the layer having acral part. When vaccine adjuvant is contained in the layer located lower in the direction to bottom than the layer having acral part, the vaccine adjuvant is easy to be delivered to Langerhans cells located in the epidermal layer.

The dissolving microneedle preparation has a bottom part diameter of 30-1000 μm, preferably 150-500 μm, more preferably 200-350 μm and has an inserting direction length of 50-1500 μm, preferably 100-750 μm, more preferably 200-600 μm. When the size of dissolving microneedle preparation is out of this range, strength may become insufficient or insertion performance may decrease. More specifically, the dissolving microneedle preparation may have a cone shape of which inserting direction length is about 500 μm and bottom part diameter is about 300 μm.

The obtained dissolving microneedle preparation is used for administering vaccine antigen and vaccine adjuvant to the body of human or animals. As to administration method, at first, the acral part of dissolving microneedle is faced to the skin, the supporting body is pushed to the skin with a finger to insert the microneedle into the skin. Instead of pushing with a finger, by applying impact force, the deeper insertion may be provided.

### <Other Vaccine Preparation>

The present invention is not limited to the dissolving microneedles which are prepared by the filling method using a mold. Microneedle preparation may be prepared by coating the surface of the microneedle with dextran sulfate and vaccine antigen. For example, the object of the present invention may be achieved by coating the acral part of microneedles, which are made of metal, biodegradable or plastic polymer substances, with the dextran sulfate and the vaccine antigen.

For example, the trapezoidal shape fundamental part of the microneedle is manufactured with metal, biodegradable or plastic polymer substances, and the microneedle of the present invention may be constituted on the upper surface of the fundamental part. The acral part of such a two-layered dissolving microneedle preparation may be formed to needle shape at their acral part with a method by sticking an aqueous viscous solution containing the antigen substance, the dextran sulfate and, if necessary, the water-soluble thread-forming polymer substance, followed by pulling and drying.

Otherwise, it may be that the whole surface of microneedle is coated with the dextran sulfate or its derivative once and dried. Thereafter, surface of the skin-insertable acral part is coated with solution containing the vaccine antigen to achieve the object of the present invention.

Otherwise, the object of the present invention may be achieved by injecting vaccine antigen solution added with the dextran sulfate into the skin epidermal or dermal layer with a dermal syringe having hollow microneedles.

Otherwise, the object of the present invention may be achieved by at first making micropores reaching to the skin epidermal and/or dermal layer followed by painting the vaccine antigen solution added dextran sulfate.

### EXAMPLES

Specific embodiments are explained as follows using working examples. Of course, the present invention is not limited to the following working examples.

### Example 1

A first viscous solution (hydrogel) was prepared by adding 150 microliter of pH 7.4 phosphate buffer to 6 mg of hyaluronic acid (trade name “hyaluronic acid FCH-SU”, Kikkoman Biochemifa Company, average molecular weight 50,000-110,000), 12 mg of macromolecule dextran (trade name “DEXTRAN 70”, Meito Sangyo Co., Ltd. average molecular weight 70,000) and 5 mg of ovalbumin (trade name “OVALBUMIN”, Sigma-Aldrich Inc., St. Louis, Mo., USA) and mixing.

This viscous solution was applied on a female mold which had 300 reverse cone shaped small holes per 1 cm², about 500 micron meters in depth and about 300 micron meters diameter at the opening under a pressure condition to fill into the female mold, and it was dried.

A second viscous solution was prepared by adding 2.1 mL of pH 7.4 phosphate buffer to 600 mg of sodium chondroitin sulfate (trade name “Chondroitin sulfate C sodium”, Nacalai Tesque Co., Ltd.), 2.4 g of dextran sulfate (trade name “Sodium dextran sulfate”, Nacalai Tesque Co., Ltd. average molecular weight 5000-6000), and mixing.

A circular supporting basis having a diameter of about 1.5 cm and a thickness of about 2 mm was manufactured by tableting a mixture of cellulose acetate and hydroxypropyl cellulose in the weight ratio of 100:10.

One side of the supporting basis was applied with the second viscous solution, and was covered onto the female mold, and was dried under positive pressure. After 6 hours, the supporting basis was separated from the female mold to obtain a microneedle array chip on which 300 microneedles were formed in the shape of array.

### Comparative Example 1

A microneedle array chip was obtained according to the same manner as described in Example 1 except that a second viscous solution was prepared with 2.4 g of macromolecule dextran instead of the dextran sulfate.

### Example 2

A first viscous solution (hydrogel) was prepared by adding 200 microliter of pH 7.4 phosphate buffer to 6 mg of sodium chondroitin sulfate (trade name “Sodium chondroitin sulfate C”, Nacalai Tesque Co., Ltd.), 24 mg of dextran sulfate (trade name “Sodium dextran sulfate”, Nacalai Tesque Co., Ltd., average molecular weight 5,000-6,000) and 5 mg of ovalbumin (trade name “Ovalbumin”, Sigma-Aldrich Inc., St. Louis, Mo., USA) and mixing.

This viscous solution was applied on the female mold which had 300 reverse cone shaped small holes per 1 cm², about 500 micron meters in depth and about 300 micron meters diameter at the opening under a pressure condition to fill into the female mold, and it was dried.

A second viscous solution was prepared by adding 2.1 mL of pH 7.4 phosphate buffer to 600 mg of sodium chondroitin sulfate (trade name “Chondroitin sulfate C sodium”, Nacalai Tesque Co., Ltd.), 1.2 g of macromolecule dextran (trade name “Dextran 70”, Meito Sangyo Co., Ltd. average molecular weight 70,000) and mixing.

A circular supporting basis having a diameter of about 1.5 cm and a thickness of about 2 mm was manufactured by tableting a mixture of cellulose acetate and hydroxypropyl cellulose in the weight ratio of 100:10.

One side of the supporting basis was applied with the second viscous solution, and was covered onto the female mold, and was dried under positive pressure. After 6 hours, the supporting basis was separated from the female mold to obtain a microneedle array chip on which 300 microneedles were formed in the shape of array.

### Example 3

Efficacy of the skin vaccine was evaluated using Wistar rats having a body weight of about 230 g. Either preparation in Example 1 or Comparative example 1 was administered to the hair removed rat skin by inserting a chip. The administrations were conducted 2 times, on day 0 and on day 14, and blood samplings were conducted on day 14 and on day 28. Total antibody titer Ig (G+A+M) was measured using the obtained blood samples.

The total antibody titer Ig (G+A+M) on 14th day after the first administration of the preparation were 15.1±2.6×10⁴ U/mL for the Example 1 preparation and 3.1±0.7×10⁴ U/mL for the Comparative example 1 preparation.

The total antibody titer Ig (G+A+M) on 28th day after the first administration of the preparation were 310.2±47.3×10⁴ U/mL for the Example 1 preparation and 143.3±35.7×10⁴ U/mL for the Comparative example 1 preparation.

In the dissolving microneedle preparation of Example 1 in which dextran sulfate was used as the base, higher antibody titer was induced than the microneedle preparation of Comparative example 1 in which conventional water-soluble and thread-forming polymer substance was used as the base.

### Example 4

A first viscous solution was prepared by adding 520 microliter of phosphate buffer, pH 7.4, to 20 mg of hyaluronic acid (trade name “Hyaluronic acid FCH-SU”, Kikkoman BioChemifa Co., Ltd, average molecular weight 50,000-110,000), 40 mg of macromolecule dextran (trade name “Dextran 70”, Meito Sangyo Co., Ltd. average molecular weight 70,000), 4.0 mg of brilliant blue, labelling dye, and mixing. This viscous solution was applied on a female mold which had 300 reverse cone shaped small holes per 1 cm², about 500 micron meters in depth and about 300 micron meters diameter at the opening under a pressure condition to fill into the female mold, and it was dried.

A second viscous solution was prepared by adding 1.5 mL of phosphate buffer, pH 7.4, to 600 mg of sodium chondroitin sulfate (trade name “Sodium chondroitin sulfate C”, Nacalai Tesque Co., Ltd.), 2.4 g of dextran sulfate (trade name “Sodium dextran sulphate”, Nacalai Tesque Co., Ltd.), and mixing.

A circular supporting basis having a diameter of about 1.5 cm and a thickness of about 2 mm was manufactured by tableting a mixture of cellulose acetate and hydroxypropyl cellulose in the weight ratio of 100:10.

One side of the supporting basis was applied with the second viscous solution, and was covered onto the female mold, and was dried under positive pressure. After 6 hours, the supporting basis was separated from the female mold to obtain a microneedle array chip on which 300 microneedles were formed in the shape of array.

### Comparative Example 2

A viscous solution was prepared by adding 1.0 mL of phosphate buffer, pH 7.4, to 440 mg of hyaluronic acid (trade name “Hyaluronic acid FCH-SU”, Kikkoman Biochemifa Co. Ltd., average molecular weight 50,000-110,000), 880 mg of macromolecule dextran (trade name “Dextran 70”, Meito Sangyo Co., Ltd. average molecular weight 70,000 and mixing.

A microneedle array chip was obtained according to the same manner as described in Example 4, except that the viscous solution was used instead of the second viscous solution containing sodium chondroitin sulfate and dextran sulfate.

### Example 5

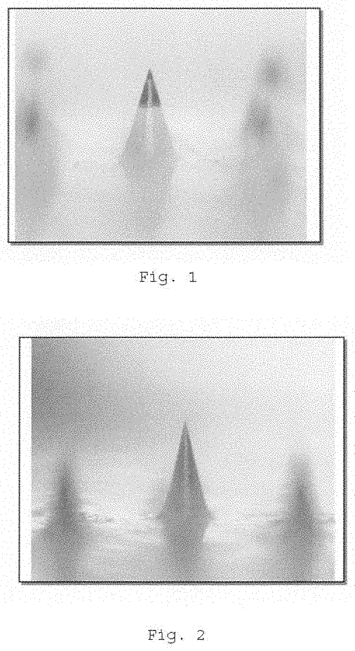

The dissolving microneedles obtained in Example 4 and Comparative example 2 were observed by a video microscope made by Keyence Co., Ltd., and the results were shown in FIG. 1 and FIG. 2.

For the microneedles obtained with Example 4, acral parts are labeled with blue color due to the presence of brilliant blue and the interface between the fundamental part is clearly shown. The length of the acral part labeled with blue color was measured to be 148 μm. On the other hand, for the microneedle obtained with Comparative example 2, they were labeled with blue color equally from the acral part to the fundamental part, because the brilliant blue filled in the acral part diffused to the fundamental part.

### Example 6

A first viscous solution (hydrogel) was prepared by adding 60 microliter of pH 7.4 phosphate buffer to 24 mg of dextran sulfate (trade name “Sodium dextran sulfate”, Nacalai Tesque Co., Ltd., average molecular weight 5,000-6,000), 5 mg of ovalbumin (trade name “Ovalbumin”, Sigma-Aldrich Inc., St. Louis, Mo., USA) and mixing.

This viscous solution was applied on a female mold which had 300 reverse cone shaped small holes per 1 cm², about 500 micron meters in depth and about 300 micron meters diameter at the opening under a pressure condition to fill into the female mold, and it was dried.

A second viscous solution was prepared by adding 1.0 mL of pH 7.4 phosphate buffer to 600 mg of sodium chondroitin sulfate (trade name “Sodium chondroitin sulfate C”, Nacalai Tesque Co., Ltd.), 600 mg of macromolecule dextran (trade name “Dextran 70”, Meito Sangyo Co., Ltd. average molecular weight 70,000), and mixing.

A circular supporting basis having a diameter of about 1.5 cm and a thickness of about 2 mm was manufactured by tableting a mixture of cellulose acetate and hydroxypropyl cellulose in the weight ratio of 100:10.

One side of the supporting basis was applied with the second viscous solution, and was covered onto the female mold, and it dried under positive pressure. After 6 hours, the supporting basis was separated from the female mold to obtain a microneedle array chip on which 300 microneedles were formed in the shape of array.

### INDUSTRIAL APPLICABILITY

The present invention enables to produce dissolving microneedle preparation having high strength, solubility and high immunological activity, and is useful in industry.

1-11. (canceled)

12. A dissolving microneedle preparation containing dextran sulfate or its derivatives as base.

13. The dissolving microneedle preparation according to claim 12 further containing a vaccine antigen.

14. The dissolving microneedle preparation according to claim 12 having several layers laminated in the direction connecting between acral part and bottom part.

15. The dissolving microneedle preparation according to claim 14, wherein the layer having acral part contains a vaccine antigen.

16. The dissolving microneedle preparation according to claim 14, wherein the layer having bottom part does not contain a vaccine antigen.

17. The dissolving microneedle preparation according to claim 14, wherein a layer, which is different from the layer containing vaccine antigen, contains the dextran sulfate or its derivatives as base.

18. The dissolving microneedle preparation according to claim 17, wherein a layer, which is located lower in the direction to bottom than the layer containing vaccine antigen, contains the dextran sulfate or its derivatives as base.

19. The dissolving microneedle preparation according to claim 12 containing a polymer substance having thread-forming property and water-soluble property as base.

20. The dissolving microneedle preparation according to claim 12, wherein the dextran sulfate or its derivatives is used as a vaccine adjuvant.

21. The dissolving microneedle preparation according to claim 20, wherein the dextran sulfate is contained in an amount of more than 50% by weight of the base when the mixture of dextran sulfate and water-soluble thread-forming polymer substance is used as the base.

22. The dissolving microneedle preparation according to claim 20, wherein the polymer substance having thread-forming property and water-soluble property is hyaluronic acid or sodium chondroitin sulfate.

23. The dissolving microneedle preparation according to claim 12 fixed on a supporting body.

24. A microneedle array chip having a supporting body and the dissolving microneedle preparations according to claim 12 formed in the shape of array on the supporting body.
